(19) United States
(12) Patent Application Publication
(10) Pub. No.: US 2015/0374242 A1
(43) Pub. Date: Dec. 31, 2015

(54) METHOD FOR MEASUREING BLOOD VESSEL ELASTICITY

(71) Applicant: PROLIFIC TECHNOLOGY INC., TAIPEI (TW)

(72) Inventors: Weichih Hu, TAIPEI (TW); Guan-Yu Chen, TAIPEI (TW); Tingyin Kuo, TAIPEI (TW)

(21) Appl. No.: 14/317,144

(22) Filed: Jun. 27, 2014

(51) Int. Cl. A61B 5/02; A61B 5/00; A61B 5/022

(52) U.S. Cl. CPC A61B 5/02007; A61B 5/02225; A61B 5/7278; A61B 5/742

(57) ABSTRACT

According to one embodiment of a method for measuring blood vessel elasticity, adapted to a sphygmus oscillation signal measured by pressure sensor of blood pressure equipment. This method for measuring blood vessel elasticity includes: uses the sphygmus oscillation signal to calculate systolic blood pressure and diastolic blood pressure; extracts a sphygmus signal after corresponds to the diastolic blood pressure; finds the maximum slope of the extracted sphygmus signal; finds a first time by using the maximum slope down to a reference pressure; then finds a second time by using the maximum slope down to the lowest point of the extracted sphygmus signal; and compares the first time and the second time to get the indicator of blood vessel elasticity.

### BACKGROUND

Early methods for measuring blood vessel elasticity are mainly intrusive, mostly user got negative feeling on complex and time consuming measurement process, and therefore a non-invasive way to measure the elasticity of blood vessels is developed. Recently, non-invasive way for measuring blood vessel elasticity uses the pulse wave velocity (PWV) as the standard for judging blood vessel elasticity. In order to get the PWV information, at least two sets of cuff or sensors are established to measure blood pressure in different parts of the human body synchronously, and coupled with the ECG signal as a standard reference of time. In the example of using two cuffs for measurement, these two cuffs are placed on the arm and the ankle on the body respectively for performing two simultaneous measurements, the calculation formula of PWV is following, i.e.,

$$PWV = \frac{Distance}{t} ,$$

wherein the Distance is the distance between the upper arm and the ankle, t is the time difference of waveforms. According to the PWV formula, the distance between two cuffs and the time difference of the two waveforms on the arm and the ankle must be measured in advance. Typically the PWV less than 1200 mm/sec is the normal range.

Therefore many physiological signals required to be measured, and also coupled with a set of ECG signals by using the above method, results in relatively complicated measurement process and apparatus. Thus equipments for measuring of blood vessels elasticity are not yet common. For the general public, it is not easily for measuring blood vessel elasticity to understand their vascular condition and thus early prevent cardiovascular disease.

The above mentioned calculation method of blood vessel elasticity requires time-consuming measuring process and too many apparatus, thus the cost is relatively high. To improve the above-mentioned drawback, and eliminate two cuffs placed on the arm and the ankle with a set of ECG signals for performing time-consuming measurement, the present invention provides a simple method for measuring blood vessel elasticity coupled with blood pressure measurement, thus helps fast operation and cost reduction.

### SUMMARY

The exemplary embodiments of the disclosure may provide a device and method for measuring blood pressure.

One exemplary embodiment relates to a device for measuring blood pressure, the device includes a pressure sensor, a microprocessor, and a user interface, wherein a user exerts pressure on the user's wrist by using the pressure sensor, the pressure sensor senses the pressure to produce oscillation signal; the microprocessor connects with the pressure sensor, and receives the oscillation signal to calculate vessel pulse, systolic blood pressure, and diastolic blood pressure of the user; the user interface connects with the microprocessor, and receives data of the microprocessor to inform the user.

The present invention provides a method for measuring blood vessel elasticity, adapted to general electronic sphygmomanometer. The systolic blood pressure and diastolic blood pressure are calculated based on the measured pressure signal, and then blood vessel elasticity indicator is further calculated to provide the user understanding vascular status. Comparing to too many parameters required and more complex process of proposed method for calculating blood vessel elasticity, the present invention provides a simple algorithm for calculating blood vessel elasticity indicators, thus helps easy implement and cost reduction.

One exemplary embodiment relates to a method for measuring blood vessel elasticity, adapted to blood pressure oscillation signals measured by pressure sensor of blood pressure equipment. The method for measuring blood vessel elasticity includes: uses the sphygmus oscillation signal to calculate systolic blood pressure and diastolic blood pressure; extracts a sphygmus oscillation signal after corresponds to the diastolic blood pressure; finds the maximum slope of the extracted sphygmus signal; finds a first time by using the maximum slope down to a reference pressure; then finds a second time by using the maximum slope down to the lowest point of the extracted sphygmuse signal; and compares the first time and the second time to get the indicator of blood vessel elasticity.

### BRIEF DESCRIPTION OF THE DRAWINGS

FIG. 1 illustrates the architecture for measuring blood pressure.

FIG. 2 illustrates a method for measuring blood vessel elasticity, according to an exemplary embodiment.

FIG. 3 illustrates the sphygmusoscillation signal, according to an exemplary embodiment.

FIG. 4 illustrates the sphygmus oscillation signal and the corresponding extracted sphygmus signal, according to an exemplary embodiment.

FIG. 5 illustrates a single extracted sphygmus signal, according to an exemplary embodiment.

FIG. 6 illustrates a diagram for finding maximum slope of extracted sphygmus signal to determine blood vessel elasticity indicator, according to an exemplary embodiment.

### DETAILED DESCRIPTION OF DISCLOSED EMBODIMENTS

Below, exemplary embodiments will be described in detail with reference to accompanying drawings so as to be easily realized by a person having ordinary knowledge in the art. The inventive concept may be embodied in various forms without being limited to the exemplary embodiments set forth herein. Descriptions of well-known parts are omitted for clarity, and like reference numerals refer to like elements throughout.

The exemplary embodiment in the disclosure relates to a method for measuring blood vessel elasticity, this method is applicable to general electronic sphygmomanometer. This method calculates user's systolic blood pressure, diastolic blood pressure, and vessel pulse based on the outputted pressure signals of a cuff, and calculates an indicator of blood vessel elasticity to provide the user understanding their blood vessel status. Wherein a simple algorithm is proposed to calculate the indicator of blood vessel elasticity. The algorithm performs determination based on pulse signal obtained in the measurement process of blood pressure. Since the pulse signal reflects the blood vessel status, the indicator can be directly calculated by the pulse signal.

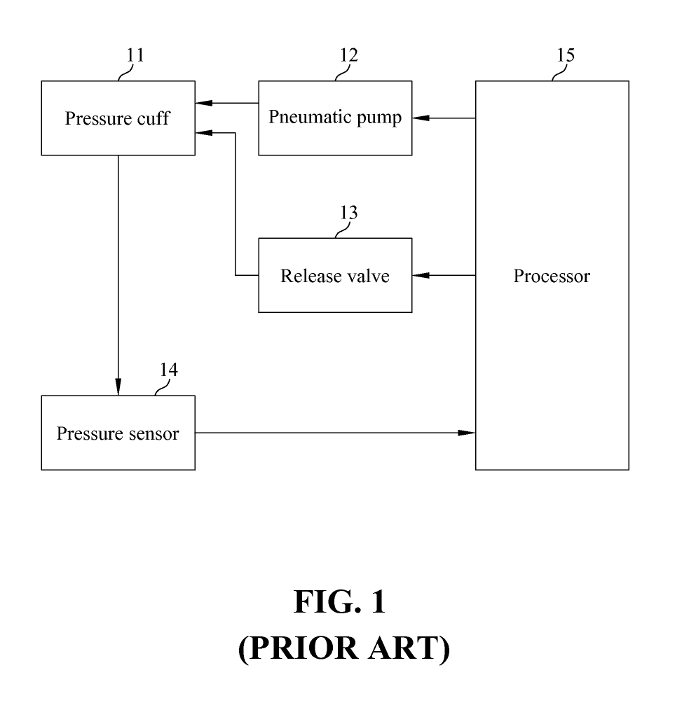

FIG. 1 illustrates the architecture for measuring blood pressure. Refer to FIG. 1, the architecture includes a pressure cuff 11, a pneumatic pump 12, a release valve 13, and a pressure sensor 14. Wherein the pressure cuff 11 is placed on upper arm of a user, the pneumatic pump 12 connects to the pressure cuff 11 and the release valve 13 connects to the pressure cuff 11 to discourage pressure, the pressure sensor 14 is provided in the pressure cuff 11 to sense the pressure to produce oscillation signal. The architecture for measuring blood pressure in FIG. 1 may also include a processor 15 for receiving the oscillation signals generated by the pressure sensor to perform calculation to obtain vessel pulse, systolic blood pressure (SBP), and diastolic blood pressure (DBP). In FIG. 1, the processor 15 may further transmit inflate or deflate control signals to the pneumatic pump 12 and the release valve 13 to control the pneumatic pump 12 and release valve 13 performing inflation and deflation of the pressure cuff 11.

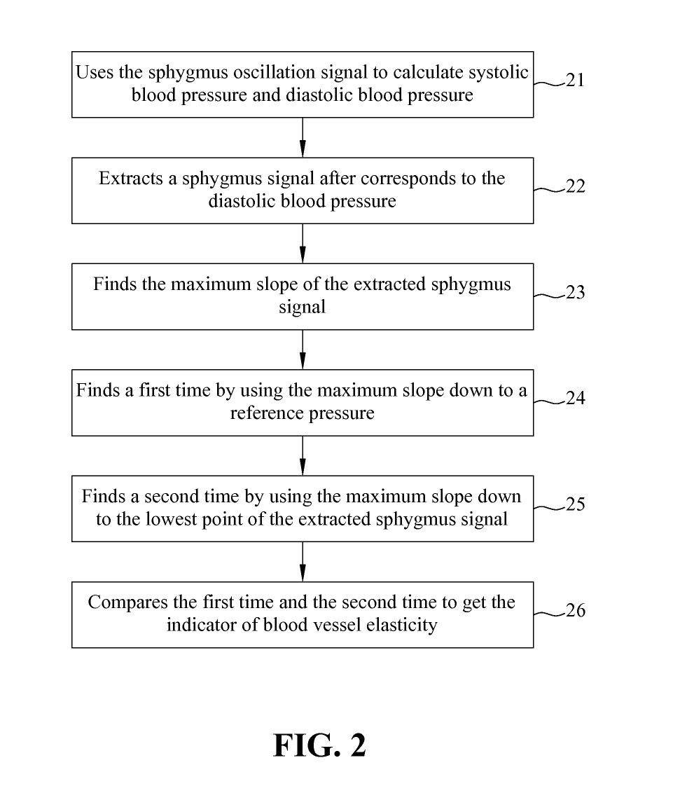

FIG. 2 illustrates a method for measuring blood vessel elasticity, according to an exemplary embodiment. This method for measuring blood vessel elasticity is suitable for oscillation signal measured by pressure sensor in sphygmomanometer. The sphygmomanometer is such as, but is not limited to the architecture in FIG. 1. Refer to FIG. 2, the method for measuring blood vessel elasticity includes: uses the sphygmus oscillation signal to calculate systolic blood pressure and diastolic blood pressure (step 21); extracts a sphygmus oscillation signal after corresponds to the diastolic blood pressure (step 22); finds the maximum slope of the extracted sphygmus signal (step 23); finds a first time by using the maximum slope down to a reference pressure (step 24); then finds a second time by using the maximum slope down to the lowest point of the extracted sphygmus signal (step 25); and finally, compares the first time and the second time to get the indicator of blood vessel elasticity (step 26).

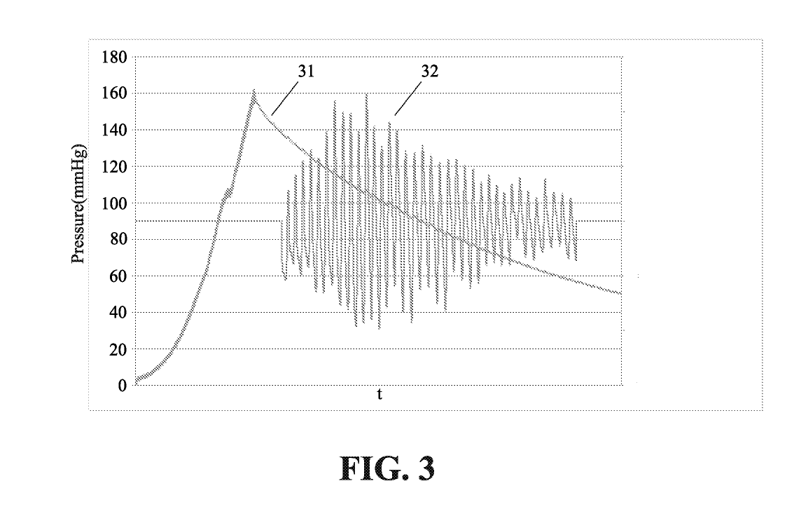

In the step 21 of FIG. 2, the method uses the sphygmus oscillation signal to calculate systolic blood pressure and diastolic blood pressure. As mentioned above, the sphygmus oscillation signals are the signals measured by the pressure sensor in sphygmomanometer. FIG. 3 illustrates the oscillation sphygmus signal, according to an exemplary embodiment, wherein the horizontal axis represents time and the vertical axis represents pressure. The line 31 of slowly up and slowly down in FIG. 3 is the cuff pressure of the phygmomanometer, and the rapid changing waveform 32 in FIG. 3 is the sphygmus oscillation signal measured by the pressure sensor in the sphygmomanometer. Each up and down of the sphygmus oscillation signal represents of a pulse beat fluctuation, so that the vessel pulse value may thus be calculated by the oscillation signals. In addition, the corresponding pressure value of the maximum amplitude of the oscillation signal is the average blood pressure value. In FIG. 3, the corresponding pressure value of the maximum amplitude is 110 mmHg, e.g., the average blood pressure value is 110 mmHg Then the systolic blood pressure and the diastolic blood pressure are calculated base on this average pressure value.

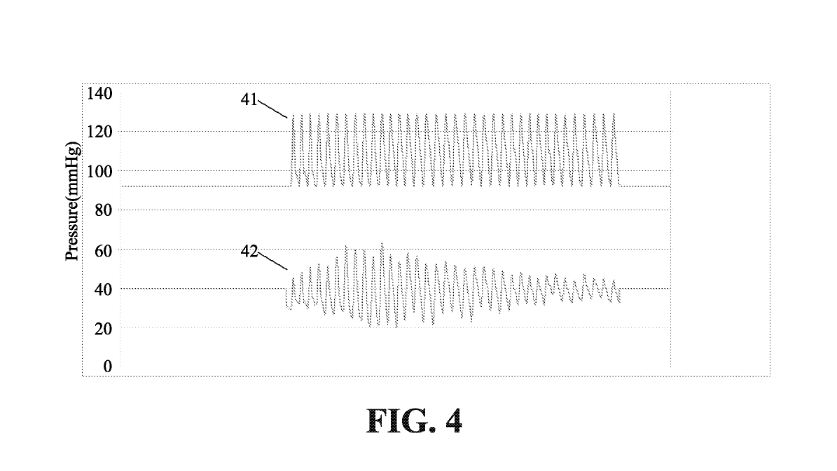

Following the above, in the step 22 in FIG. 2, the method transfers the sphygmus oscillation signal in step 21 into corresponding pulse wave, and extracts a sphygmus signal after corresponds to the diastolic blood pressure, wherein the sphygmus oscillation signal and the corresponding pulse wave are shown in FIG. 4. In FIG. 4, the inconsistent amplitude signal 42 is the sphygmus oscillation signal, which is the corresponding sphygmus oscillation signal in FIG. 3. The top of the sphygmus oscillation signal represents the systolic blood pressure; the low one represents diastolic blood pressure. In FIG. 4, the signal 41 is the corresponding pulse wave for extracted sphygmus signal.

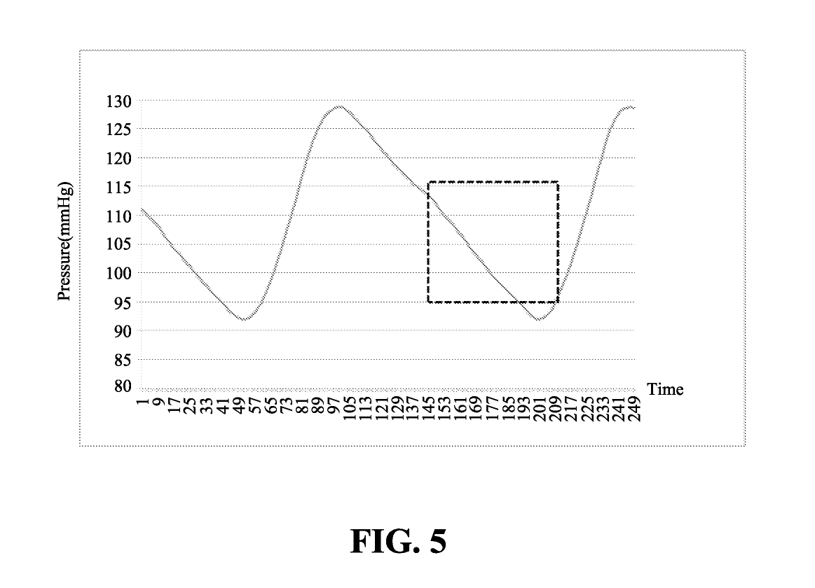

FIG. 5 illustrates a single extracted sphygmus signal according to an exemplary embodiment, which is one of the corresponding pulse wave shown in FIG. 4. This single pulse wave is selected after corresponds to the diastolic blood pressure.

The method for calculating indicator of blood vessel elasticity provided in the disclosure may figure out blood vessel elasticity based on the extracted sphygmus signal in FIG. 5. Therefore the corresponding falling waveform from the systolic blood pressure to the diastolic blood pressure represents the rebound speed of the blood vessel, the steeper the slope, the faster the rebound speed of the blood vessel, while the slowly decline of the slope, the slower the rebound speed of the blood vessel.

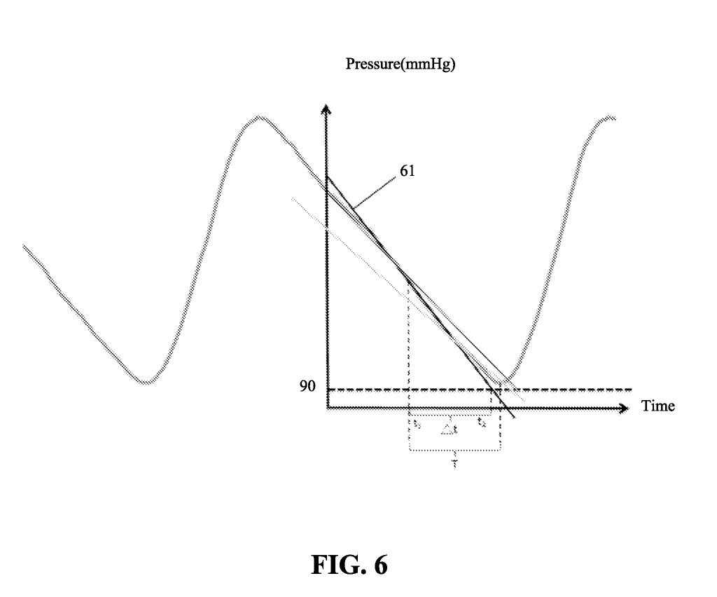

Following the above, the step 23 of the method for measuring the blood vessel elasticity may find the maximum slope of the extracted sphygmus signal to determine the indicator of blood vessels elasticity, such as shown in FIG. 6. Then the step 24 of the method for measuring the blood vessel elasticity may find a first time by using the maximum slope (the solid line 61 in FIG. 6) down to a reference pressure. Wherein the reference pressure may be any pressure, for example, may be 90 mmHg (the high diastolic blood pressure defined by World Health Organization), and when the blood pressure is high, the blood vessel wall may easily be injured due to high withstand pressure to form atherosclerosis, and thus the reference pressure may be set to 90 mmHg. The first time required is the difference Δt of t1 and t2 in FIG. 6.

Then the method may find a second time T by using the maximum slope down to the lowest point of the extracted sphygmus signal, as shown in FIG. 6. Finally, the first time Δt is compared with the second time T to get the blood vessel elasticity indicator. For example, if Δt is less than T, means that the blood vessel have also been resting when reduced to the diastolic blood pressure, therefore the rebound speed of the blood vessel is high when the blood vessel contracts again after taking rest; If Δt≈T, means no resting before the blood vessel contracts; Finally, if Δt is greater than T, means that the blood vessel starts contraction before complete recovery, that is the rebound speed of the blood vessel is slow. Therefore, the relationship between T and Δt may used to learn the hardened condition of the blood vessel.

The method for measuring blood vessels elasticity of the present invention may further perform signal filtering and calculation for the oscillation signal to calculate the systolic blood pressure and diastolic blood pressure of the user. The method for measuring blood vessel elasticity of the present invention may also display the blood vessel elasticity indicator to the user for reference, such as display systolic blood pressure, diastolic blood pressure, vessel pulse, and indicator of blood vessel elasticity on a monitor.

In summary, the method for calculating the blood vessel elasticity indicator may calculate the blood vessel elasticity indicator bases on vessel pulse signal to provide the user as a reference. And the combination of this method to the general electronic sphygmomanometer allows users to quickly learn the indicator of blood vessel elasticity through blood pressure measurement process, and thus may be popularized used to effectively prevent cardiovascular disease.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A device for measuring blood vessel elasticity, adapted to sphygmus oscillation signal measured by pressure sensor of blood pressure equipment, comprising:
uses said sphygmus oscillation signal to calculate systolic blood pressure and diastolic blood pressure;
extracts a sphygmus signal after corresponds to said diastolic blood pressure;
finds maximum slope of said extracted sphygmus signal;
finds a first time by using said maximum slope down to a reference pressure;
finds a second time by using said maximum slope down to lowest point of said extracted sphygmus signal; and
compares said first time and said second time to get indicator of blood vessel elasticity.

2. The method as claimed in claim 1, wherein said reference pressure is 90 mmHg.

3. The method as claimed in claim 1, wherein said method further includes performs signal filtering and calculation for said oscillation signal to calculate said systolic blood pressure and said diastolic blood pressure.

4. The method as claimed in claim 1, wherein said method further includes displays said indicator of blood vessel elasticity to a user for reference.
